(19) United States
(12) Patent Application Publication
(10) Pub. No.: US 2016/0354256 A1
(43) Pub. Date: Dec. 8, 2016

(54) APPARATUS FOR DISTRIBUTING PARTICULATE MATERIAL AND MACHINE FOR MAKING ABSORBENT SANITARY ARTICLES COMPRISING THE APPARATUS

(71) Applicant: GDM S.P.A., Bologna (IT)

(72) Inventors: Matteo PIANTONI, Albino (Bergamo) (IT); Valerio SOLI, Bologna (IT)

(21) Appl. No.: 15/112,932

(22) PCT Filed: Feb. 9, 2015
(86) PCT No.: PCT/IB2015/050957

§ 371 Date: Jul. 20, 2016

(30) Foreign Application Priority Data
Feb. 7, 2014 (IT) BO2014A000057

(51) Int. Cl. A61F 13/15; B05C 19/06; B05C 19/04; B05B 7/14; B05B 12/08

(52) U.S. Cl. CPC A61F 13/15634; A61F 13/15658; A61F 13/15747; B05B 7/1445; B05B 12/081; B05C 19/04; B05C 19/06

(57) ABSTRACT

An apparatus for distributing particulate material on a conveyor movable along a respective operating direction includes: a tank containing particulate material and equipped with feed means for feeding the particulate material; and distribution means located downstream of the tank to receive the particulate material and the particulate material on the conveyor. The distribution means include a diffuser element and a vibrating device to shake the diffuser element. The diffuser element extends with its sliding plane along its axis between an infeed portion to receive a preset flow quantity “Q” of particulate material, and an outfeed portion. The diffuser element includes a transversal partition at a predetermined distance from the sliding plane to form a neck which acts in combination with a vibration imparted by the vibrating device to distribute the particulate material along a predetermined width of the outfeed portion.

### TECHNICAL FIELD

This invention relates to an apparatus for distributing particulate material and to a machine for making absorbent sanitary articles, such as baby nappies, sanitary towels or the like.

More specifically, this invention applies to the manufacture of sanitary underwear such as baby nappies, sanitary towels or the like.

### BACKGROUND ART

As is known, nappies comprise an absorbent pad or padding which is normally enclosed between a permeable inner layer of non-woven fabric and an impermeable outer layer of polyethylene.

Absorbent pads of known type comprise an absorbent core made of an absorbent material, such as, for example, granules of superabsorbent polymer material (SAP) inside a mixture of containment cellulose pulp (fluff) and absorbent material binder, sandwiched between two layers of non-woven fabric.

In order to improve drainage of the absorbed liquid and, at the same time, reduce production costs, manufacturers are opting for absorbent cores with more and more granular superabsorbent material and less and less cellulose pulp, and even absorbent cores that are totally free of cellulose.

One example of absorbent pads comprising absorbent cores made of superabsorbent polymer material and substantially free of cellulose is described in document EP2444046.

The padding described in that prior art document comprises a first layer of non-woven fabric, a second layer of non-woven fabric and the cellulose free absorbent material enclosed between the two layers of non-woven fabric.

The two layers of non-woven fabric are joined by seals which are suitably made in such a way as to form a plurality of cells containing the absorbent material.

In one embodiment, the seals which separate the cells are uninterrupted so as to better contain and position the absorbent material, especially when the product is new, unused and the superabsorbent material dry.

In a second embodiment, the seals have suitable breaks in them so as to improve the diffusion of the liquid and optimize its absorption by the absorbent material.

These solutions, where the absorbent core is cellulose free and the padding thus less coherent, have some disadvantages, however.

First of all, the discretization of the cells containing the absorbent material greatly accentuates problems of non-uniformity, if any, in the distribution of the absorbent (particulate) material inside the cells.

An excessive quantity of material in a cell might cause the particulate material to be forced out of the space designed to contain it, breaking the seals which separate the cells from each other and by which the two layers of non-woven fabric are joined to each other.

Disadvantageously, these problems on the one hand, reduce the absorption efficacy of the sanitary article and, on the other, have a negative effect on the structure, leading to dimensional disparity between the cells and failure of the two layers to remain sealed to each other.

Moreover, current machines for making absorbent sanitary articles are designed to make pads (and absorbent articles) which, during production, are oriented along the direction of movement of the webs (or operating direction).

Obviously, this places a strong limitation on the machines concerned in terms of productivity, which can only be increased by enhancing the performance of the devices in terms of speed and/or frequency, which, as is known, is anything but easy to obtain.

### DISCLOSURE OF THE INVENTION

In this context, the main technical purpose of this invention is to overcome the above mentioned disadvantages of the prior art by proposing an apparatus for distributing particulate material, preferably absorbent polymer material, and a machine for making absorbent sanitary articles comprising the apparatus.

More precisely, this invention has for an aim to propose an apparatus for distributing particulate material of absorbent polymer type and a machine for making absorbent sanitary articles and which are capable of guaranteeing uniform distribution of the absorbent material.

More precisely, the aim of this invention is to provide a machine for making absorbent sanitary articles capable of precisely dosing the quantity of absorbent material into the individual containment cells.

The technical purpose and aims specified are substantially achieved by an apparatus for distributing particulate material, preferably absorbent polymer material (SAP), according to claim 1 and by a machine for making absorbent sanitary articles according to claim 10.

More specifically, the distribution apparatus according to this invention comprises at least: a tank containing particulate material and equipped with feed means for feeding the particulate material; and distribution means located downstream of the tank to receive the particulate material from the feed means and configured to distribute the particulate material on the drum or conveyor.

According to one aspect of this invention, the distribution means comprise at least a diffuser element and a vibrating device configured to shake the diffuser element, where the diffuser element extends with its sliding plane along its axis between an infeed portion of it, facing the feed means to receive a preset flow quantity of particulate material, and an outfeed portion of it, of predetermined width, which can be positioned to face the drum or conveyor; the diffuser element being equipped, at an intermediate position between the infeed portion and the outfeed portion, with at least one transversal partition at a predetermined distance from the sliding plane in order to form a neck which acts in combination with a vibration imparted by the vibrating device in such a way as to distribute the particulate material along the predetermined width.

In other words, the diffuser element is shaped in such a way as to uniformly disperse the feed front of the particulate material transversely to the direction of movement thereof (that is, transversely to the axis of extension of the diffuser element itself) thanks to the joint action of the vibrating device and the transversal partition.

The diffuser element is thus configured to act on the feed front in such a way as to increase its width to a value equal to the main dimension of the opening while keeping the flow quantity substantially unchanged.

Advantageously, the particulate material, that is, the absorbent polymer material, can thus be distributed uniformly across the full width of the web of non-woven fabric, preventing cells of different density from being created during subsequent operations of grouping and confining the material.

It should be noted that the distribution apparatus is installed in a machine for making absorbent sanitary articles comprising at least an impermeable outer layer and an absorbent pad superposed on the outer layer, where the machine comprises at least a feed line for feeding the web constituting the outer layer and a unit for forming the absorbent pad.

The forming unit in turn comprises first feed means for feeding a first web of non-woven fabric along the operating direction, second feed means for feeding a second web of non-woven fabric, a coupling station where the first web is coupled to the second web of non-woven fabric, and a dosing and depositing station which is operatively located upstream of the coupling station and where at least one distributed quantity of absorbent polymer material (or particulate material) is placed on the first web of non-woven fabric.

According to one aspect of the invention, the dosing and depositing station comprises an apparatus for distributing particulate material as described in the foregoing, where the particulate material is defined by the aforementioned absorbent polymer material.

The first feed means, on the other hand, are defined, preferably, by a drum or conveyor located downstream of the distribution apparatus.

This advantageously guarantees uniform distribution of the absorbent polymer material across the full width, that is, the dimension at right angles to the operating direction, of the first web.

More precisely, the dimension of the drum or conveyor transversal to the operating direction has a portion which is shaped to receive the particulate material distributed by the distribution apparatus.

Preferably, this working portion of the transversal dimension of the drum or conveyor is equal to the predetermined width of the outfeed portion of the diffuser element.

In order to make the cells of polymer material and guarantee their uniformity, the drum or conveyor has a plurality of cavities formed along its periphery (facing the outfeed portion of the diffuser element) and suction means associated with the cavities to produce a negative pressure at the cavities themselves so that each cavity holds down a portion of the first web to form a pocket (or zone) for receiving the absorbent polymer material.

This advantageously guarantees that the same quantity of absorbent polymer material is sucked into each cavity, that is, into each pocket.

### BRIEF DESCRIPTION OF THE DRAWINGS

Further features and advantages of this invention are more apparent in the description below, with reference to preferred, non-limiting embodiments of an apparatus for distributing particulate material, preferably absorbent polymer, and of a machine for making absorbent sanitary articles, as illustrated in the accompanying drawings, in which:

FIG. 1 is a schematic side view illustrating a first embodiment of a machine for making absorbent sanitary articles according to this invention, during use;

FIG. 2 is a schematic front cross section of the machine of FIG. 1;

FIGS. 3a and 3b show a detail from FIG. 2 in two successive operating steps;

FIG. 4 is a front view of a detail of an apparatus according to this invention for distributing particulate material;

FIG. 5 shows a side view of the detail of FIG. 4;

FIG. 6 is a schematic side view illustrating a second embodiment of a machine for making absorbent sanitary articles according to this invention, during use;

FIG. 7 is a schematic front cross section of the machine of FIG. 6;

FIG. 8 is a part perspective view illustrating a second embodiment of an apparatus for distributing particulate material according to the invention, in a non-operating condition;

FIG. 9 is a front view of a detail of the apparatus of FIG. 8 for distributing particulate material;

FIG. 10 shows a side view of the detail of FIG. 8;

FIG. 11 shows a plan view of the detail of FIG. 10;

FIG. 12 shows a transversal cross section of a diffuser element for the apparatus of FIG. 8 for distributing particulate material.

### DETAILED DESCRIPTION OF PREFERRED EMBODIMENTS OF THE INVENTION

With reference to the accompanying drawings, the numerals 110, 210 denote an apparatus for distributing particulate material, in particular absorbent polymer material, according to the invention.

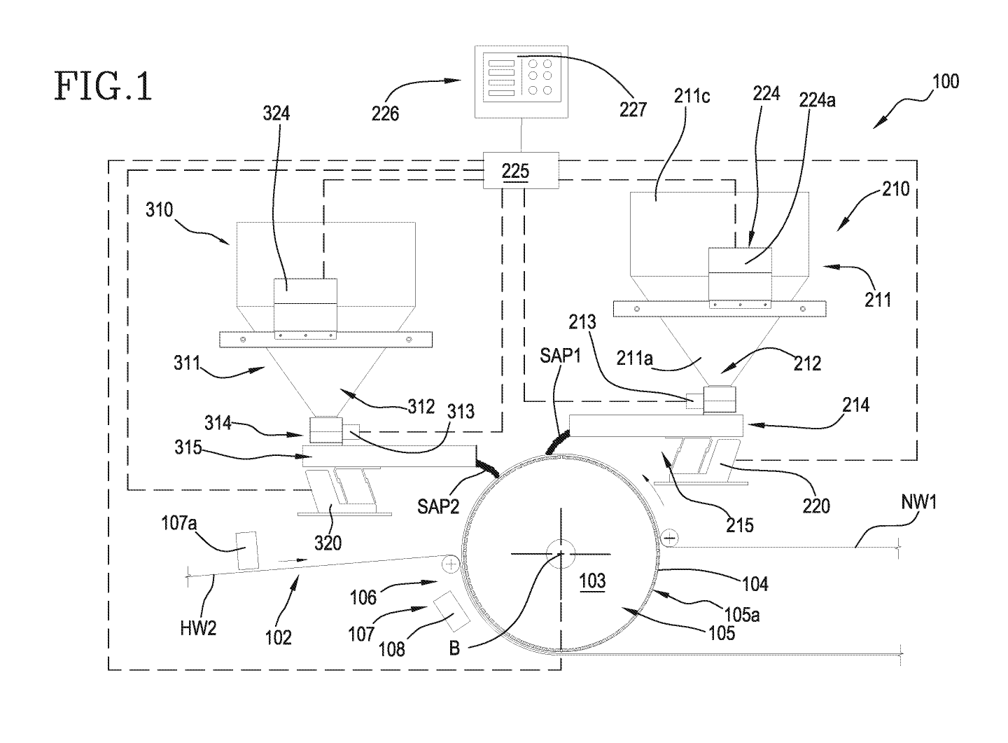

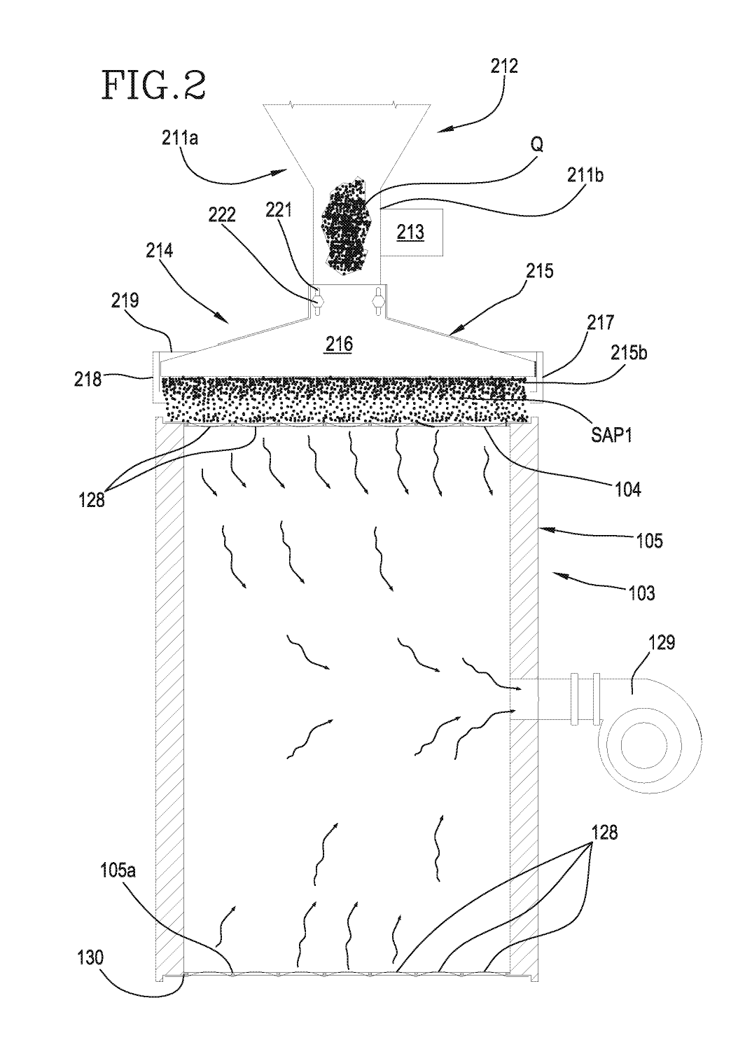

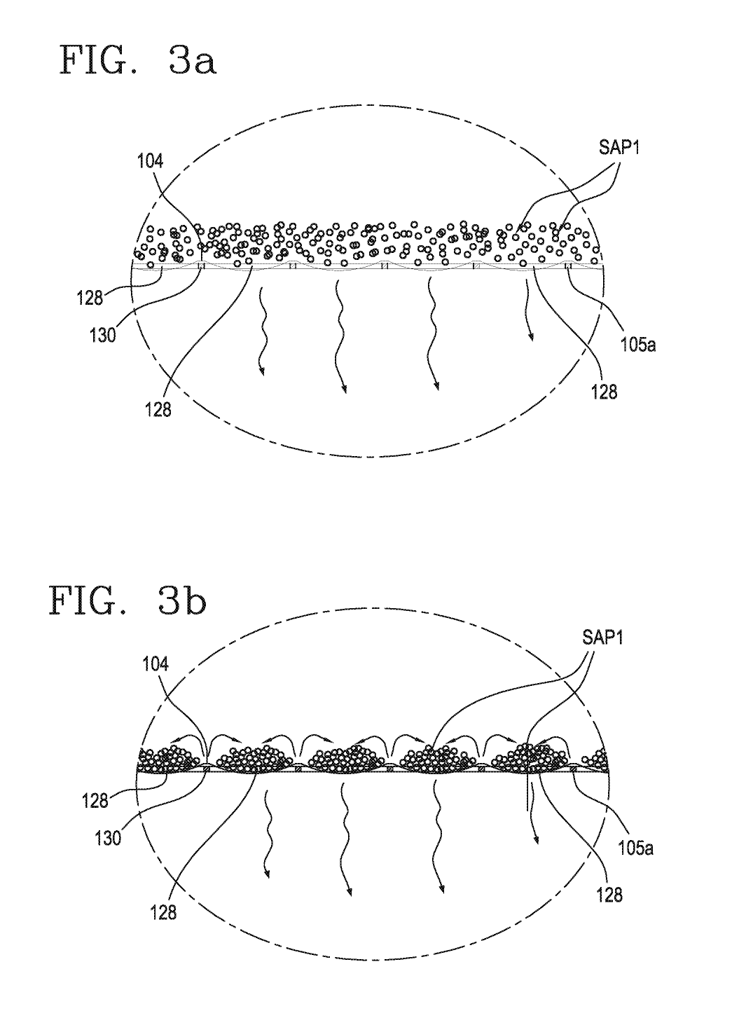

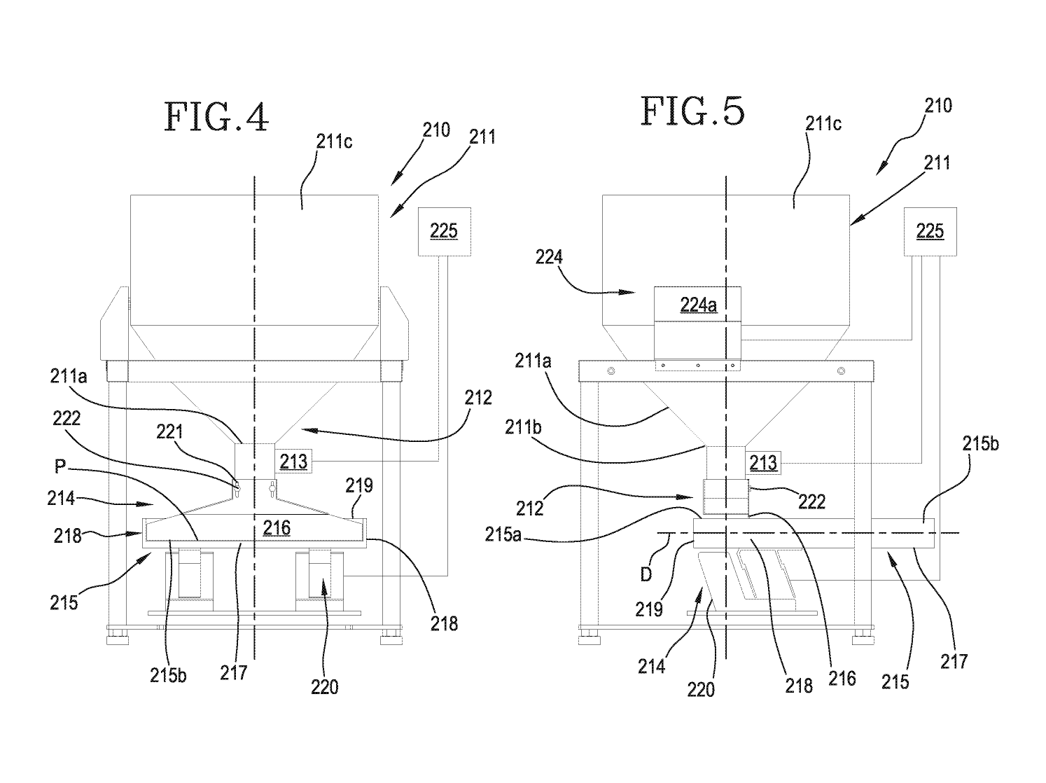

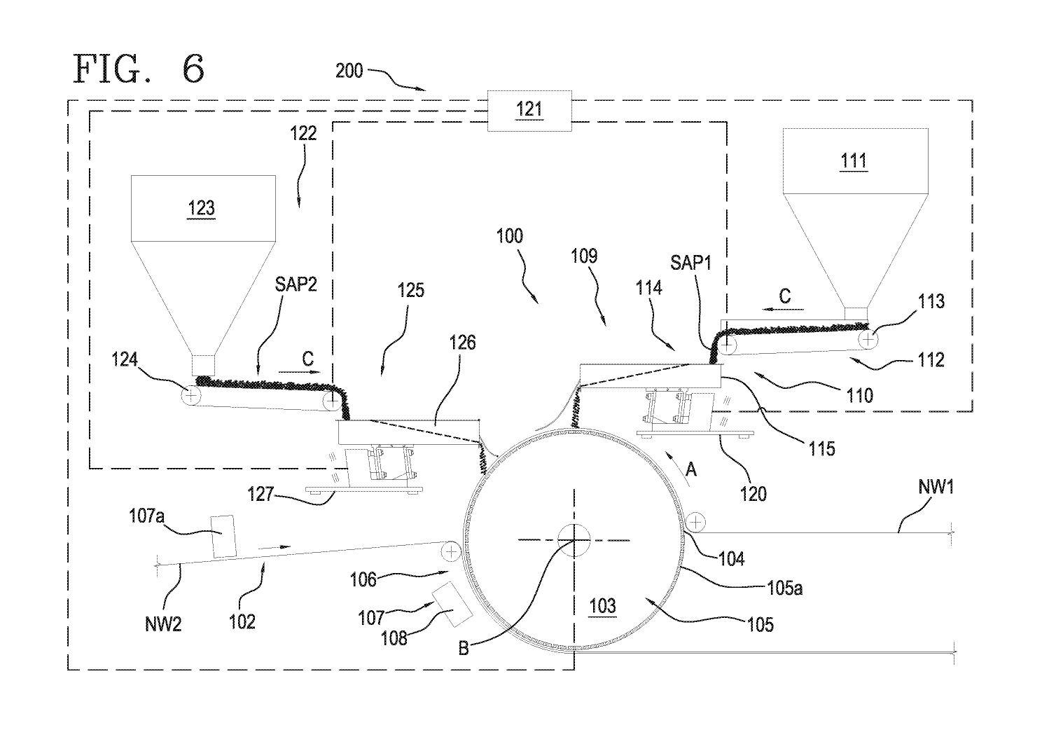

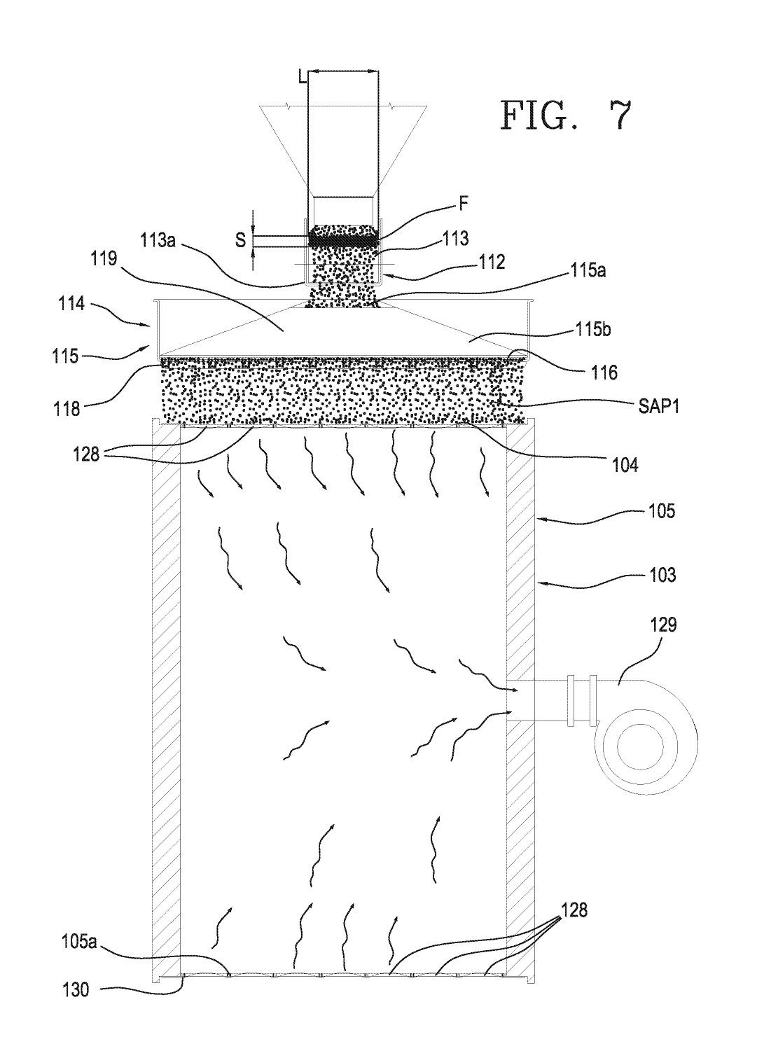

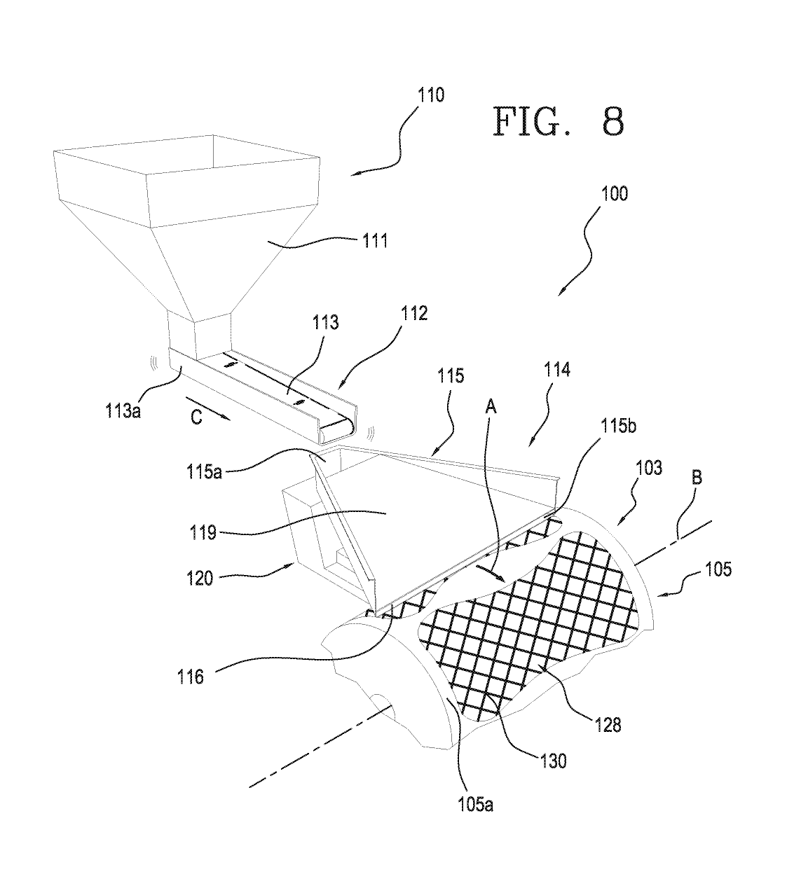

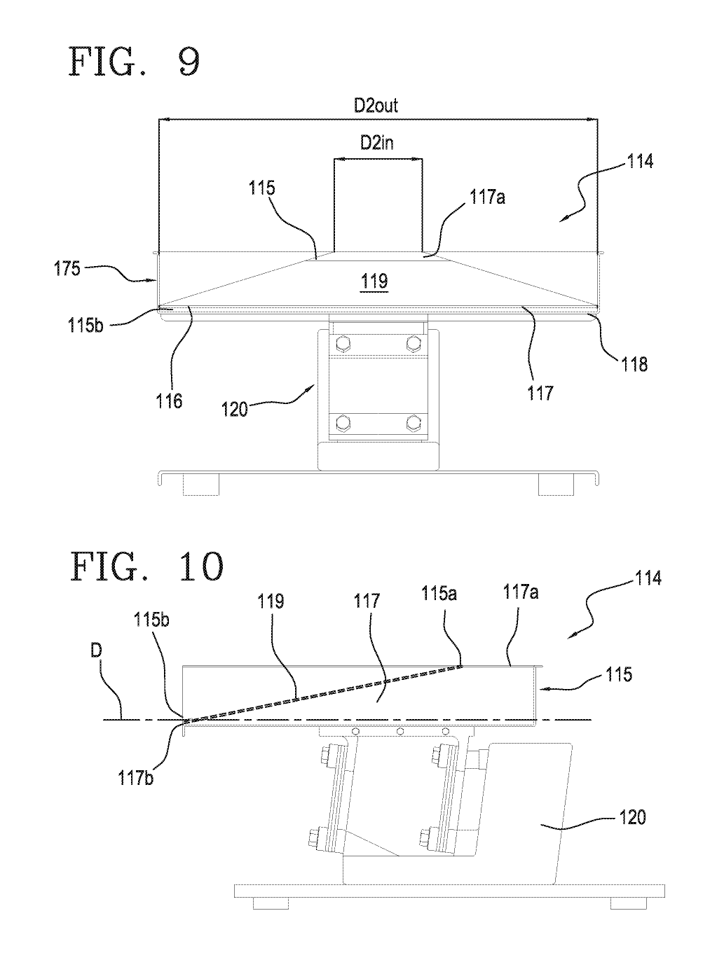

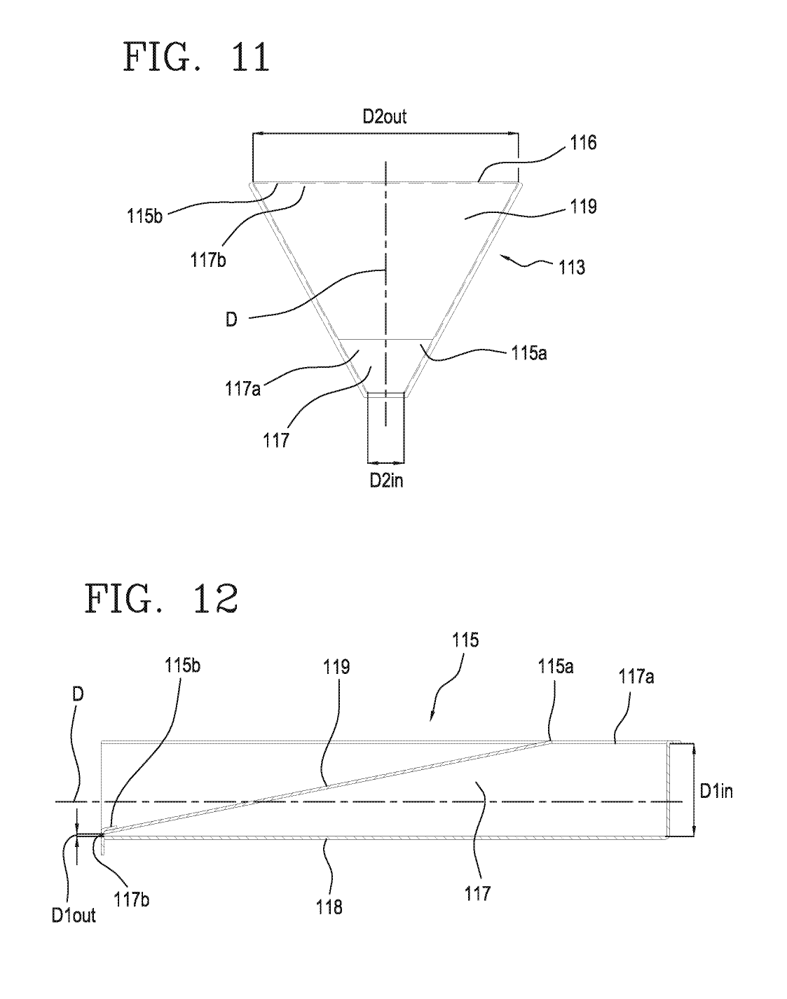

More specifically, the numeral 210 is used to refer to a first embodiment of the apparatus (FIGS. 1-5), whilst the numeral 110 is used to refer to a second embodiment of the apparatus (FIGS. 6-12).

As already stressed, the apparatus 110, 210 is installed in a machine 100 for making absorbent sanitary articles such as baby nappies, sanitary towels or the like.

These absorbent sanitary articles are preferably of the type comprising at least an impermeable outer layer and an absorbent pad superposed on the outer layer, where the pad is defined by cells of absorbent polymer material, known in the jargon of the trade as SAP (Super Absorbent Polymer).

In other words, the apparatus 110, 210 according to the invention is particularly useful in and applicable to a machine for making absorbent sanitary articles of the fluffless type, that is to say, substantially free of the cellulose layer in the padding.

The machine, described only insofar as necessary for understanding this invention, comprises a feed line, not illustrated, for feeding at least one continuous web of impermeable material for defining an impermeable outer layer (not illustrated) of the absorbent sanitary article.

The machine also comprises a forming unit 100 for making the absorbent pads of the absorbent sanitary articles to make a succession of pads and associate them with the continuous web of impermeable material (at a subsequent station).

The forming unit 100 then comprises first feed means 101 for feeding a first web NW1 of non-woven fabric along a respective operating direction “A” and second feed means 102 for feeding a second web NW2 of non-woven fabric.

Preferably, the first feed means 101 comprise a drum or conveyor 103 movable along a preset operating direction “A” and defining a feed surface 104 by which the first web NW1 is moved along the operating direction “A”.

In the preferred embodiment, the feed surface 104 is a peripheral surface of the drum or conveyor 103.

Preferably, the drum or conveyor 103 is defined by a roller 105 rotatable about a respective axis of rotation “B” transversal (that is, at right angles) to the operating direction and having a substantially cylindrical peripheral surface 105a defining a feed surface 104.

The second feed means 102, on the other hand, are defined, again preferably, by rollers and tie rods configured to transport the second web NW2 towards the first web NW1.

In effect, the first web NW1 and the second web NW2 are superposed on each other and define a first and a second layer of the absorbent pad.

In this regard, the forming unit 100 comprises a coupling station 106 by which the first web NW1 of non-woven fabric is coupled to the second web NW2.

Preferably, the coupling station 106 comprises positioning means 106a by which the second web NW2 is placed on the first web NW1 facing the drum or conveyor 103, preferably facing the roller 105.

Thus, the positioning means 106a face the peripheral surface 105a of the roller 105 and are equipped with at least one transfer element 106b (for example, a roller) for keeping the second web NW2 tangent to the drum or conveyor 103, preferably tangent to the roller 105 while transferring it.

In the preferred embodiment, as will become clearer as this description continues, the coupling station 106 comprises a joining station 107 configured to couple the two webs NW1 and NW2 along preset connection lines.

The joining unit 107 may use chemical means (that is, adhesives) or it may comprise a sealing device.

In the preferred embodiment, the joining unit 107 comprises a sonotrode 108, substantially known, for producing sealing lines between the first web NW1 and the second NW2. The sonotrode 108 faces the peripheral surface 105a of the roller 105.

Alternatively, a gluing unit may be provided which is associated with the second feed means 102 and which is configured to spread a layer of adhesive material on the second web.

In order to make the absorbent pads, the forming unit 100 comprises a dosing and depositing station 109, which is operatively located upstream of the coupling station 107 and where at least one distributed quantity of absorbent polymer material SAP1 is placed on the first web NW1 of non-woven fabric.

According to the invention, the dosing and depositing station 109 preferably comprises an apparatus 210 for distributing particulate material.

It should be noted that, hereinafter, the particulate material and the absorbent polymer material are both referred to without distinction as SAP, since the distribution apparatus 210 is intended mainly for application to a machine 100 for making absorbent sanitary articles.

In effect, the absorbent sanitary material SAP is to all intents and purposes a particulate (that is, incoherent) material.

The apparatus 210 is configured to withdraw the particulate material, or absorbent polymer material SAP1, from at least one tank 211 for storing this material and for distributing it uniformly across the full working width of the first web NW1.

In this text, the term “working width” is used to mean the dimension of the first web NW1 at right angles to the feed direction “A” and to the extent of it which receives the absorbent polymer material SAP1.

In use, therefore, the working width of the first web NW1 (which corresponds to the working width of the drum or conveyor 103) is the part of the width covered by the particulate material, that is, the absorbent polymer material SAP1, to define the pads of the absorbent sanitary articles.

In the embodiment illustrated, the “working width” substantially corresponds to the width of the first web NW1, minus two lateral strips of preset size.

More in detail, the apparatus 210 comprises feed means 212 for feeding the particulate material “SAP1” and by which a quantity of particulate material “SAP1” is fed out of the tank 211.

More precisely, the feed means 212 are at least partly defined by a bottom portion 211a of the tank 211 having a release mouth 211b of predetermined size and configured to release the particulate material preferably by gravity.

Preferably, associated with the release mouth 211b there is at least a shutter unit 213 which can be selectively switched between a closed condition where it prevents the material from flowing out of the release mouth 211b, and an open condition where it allows the material to flow out of the release mouth 211b.

The tank 211 also has a supply mouth 211c through which the particulate material “SAP1” is fed.

Preferably, means for loading the particulate material “SAP1” are (or can be) associated with the supply mouth 211c.

It should be noted that in the preferred embodiment, weight measuring means 224 associated with the tank 211 are provided which are configured to (generate and) make available a signal representing the consumption of the particulate material “SAP1”, or the quantity of particulate material “SAP1” in the tank 211.

Also provided is a control unit 225 associated with the weight measuring means 224 and with the shutter unit 213.

The control unit 225 is designed to receive from the weight measuring means 224 a signal representing the consumption of the particulate material “SAP1” and is configured to drive the shutter unit 213 as a function of the signal representing the consumption of the particulate material “SAP1” (or the quantity of particulate material “SAP1” in the tank).

More precisely, the control unit 225 is configured to activate the shutter unit 213 (that is, move it from the open condition to the closed condition) when the signal representing the quantity of particulate material “SAP1” in the tank reaches a preset minimum value.

Operatively located downstream of the tank 211, that is, of the feed means 212, the apparatus comprises distribution means 214 configured to receive the particulate material “SAP1” from the feed means 212 and to distribute the particulate material “SAP1” on the drum or conveyor 103.

Preferably, the distribution means 214 comprise at least a diffuser element 215 and a vibrating device 220 configured to shake the diffuser element 215 in order to help widen the feed front of the particulate material “SAP1”, and hence distribute the particulate material as uniformly as possible.

In this regard, the diffuser element 215 extends with its sliding plane “P” along its axis “D” between an infeed portion 215a of it, facing the feed means 212 to receive a preset flow quantity “Q” of particulate material “SAP1”, and an outfeed portion 215b of it, of predetermined width “L”, which can be positioned to face the drum or conveyor 103.

More specifically, the infeed portion 215a is located under the tank 211, preferably facing the release mouth 211b.

The outfeed portion 215b is located just above the drum 103 or conveyor.

Preferably, the axis “D” of the diffuser element 215 is parallel to a plane of rotation of the drum 103 or conveyor (that is, parallel to the direction of movement of each pocket).

To facilitate distribution of the particulate material “SAP1”, the diffuser element 215 is equipped, at an intermediate position between the infeed portion 215a and the outfeed portion 215b, with at least one transversal partition 216 at a predetermined distance from the sliding plane “P” in order to form a neck.

This neck is a narrowing or reduction in cross section, measured transversely both to the sliding plane “P” and to the axis “D”.

The transversal partition 216, or neck, acts in combination with an oscillation (or vibration) imparted by the vibrating device 220 in such a way as to help distribute the particulate material “SAP1” along the predetermined width “L”.

It should be noted that the predetermined width “L” is greater than the width, measured in the same direction, of the release mouth 211b of the tank 211.

In the preferred embodiments, the predetermined width “L” is between 150 and 600 mm, preferably between approximately 200 and approximately 500 mm.

Preferably, the diffuser element 215 is a slab-shaped member 217, defining the sliding plane “P”, from which delimiting lateral shoulders 218 rise upwards in the direction of the tank 211.

Preferably, the lateral shoulders 218 comprise at least two side panels located at the sides of the slab-shaped member 217 and oriented along the axis “D” in diverging directions or, preferably, parallel to each other.

The lateral shoulders 218, or side panels, are joined to each other by a rear wall 219, defining a U-shaped fence whose concavity faces towards the outfeed portion 215b of the diffuser element.

Preferably, the transversal partition 216 is located in the proximity of the rear wall 219.

More precisely, the infeed portion 215a of the diffuser element is delimited between the rear wall 219 and the transversal partition 216.

It should be noted that the transversal partition 216 is positioned at a predetermined distance from the sliding plane “P”, that is, from the slab-shaped member 217. More specifically, the position of the transversal partition 216 relative to the slab-shaped member is adjustable so that this distance can be changed, and hence the flow quantity of particulate material “SAP1” regulated.

In other words, the transversal partition 216 is movably connected to the diffuser element so that its position can be adjusted towards and/or away from the sliding plane “P” as a function of a flow quantity of the particulate material “SAP1” and/or of the predetermined width “L” of the outfeed portion 215b of the diffuser element 215.

In the embodiment illustrated, the side panels 217 of the diffuser element have slots 221 made in them, into which respective pins of the transversal partition 216 are insertable and/or slidable.

Clamping means 222 (preferably nuts or screws) are also provided to allow the transversal partition 216 to be fastened in a plurality of operating positions (corresponding to a plurality of distances from the sliding plane).

Preferably, the weight measuring means 224 are associated with the tank 111 and are configured to measure the weight thereof at preset intervals in order to make available a signal representing the consumption of the particulate material “SAP1”.

More precisely, the weight measuring means 224 are also associated with the distribution means 214 in order to make available a signal representing the consumption of particulate material “SAP1” in the entire apparatus.

Advantageously, therefore, the consumption of the particulate material “SAP1” can be measured accurately and in absolute terms because each time material is released through the outfeed portion 215b of the diffuser element 215, there is a corresponding drop in the weight of the system as a whole.

in the preferred embodiments, the weight measuring means 224 comprise one or more load cells associated with the tank 211 and/or with the distribution means 214.

Thus, in a first embodiment, the weight measuring means 224 comprise at least one load cell 224a associated with, that is, connected to, the tank 211.

Alternatively, the measuring means 224 comprise at least one load cell 224a associated with, that is, connected to, both the tank 211 and the distribution means 214 (that is, the diffuser element 215).

In this embodiment, the tank 211 and the diffuser element 215 are connected and bound to each other so that the change in weight of the entire system can be measured.

Preferably, the control unit 225, which is associated with the weight measuring means 224, is functionally and operatively associated also with the vibrating device 220.

Thus, the control unit 225 is configured to receive from the weight measuring means 224 the signal representing the consumption of the particulate material “SAP1”.

The control unit 225 is also configured to drive the vibrating device 220 as a function of the signal representing the consumption of the particulate material “SAP1”.

More in detail, the control unit 225 is programmed to compare the signal representing the consumption of the particulate material “SAP1” sent by the weight measuring means 224 with a preset reference value.

In this regard, at least one user interface module 226 is provided to allow setting the reference value.

More precisely, the apparatus comprises at least a control panel 227 equipped with the user interface module 226 which, in the preferred embodiment, is a touch screen.

The control unit 225 is thus programmed to generate an increment in the power of the vibrating device 220 if the consumption of the particulate material “SAP1” (measured by the measuring means 224) is less than the preset reference value.

Similarly, the control unit 225 is programmed to generate a decrease in the power of the vibrating device 220 if the consumption of the particulate material (measured by the measuring means 224) is greater than the preset reference value.

Advantageously, this allows the flow quantity of the particulate material “SAP1” distributed on the drum 103 or conveyor to be controlled precisely and accurately.

Moreover, in the embodiments where the weight of the entire system is measured, clogging of the diffuser element 215, although unlikely, can also be prevented.

Whatever the case, the control unit 225 is preferably configured to generate an alarm signal and to stop the vibrating device 220 if the power requirement of the vibrating device 220 itself following the aforementioned comparison operation exceeds an upper limit value and/or drops below a lower limit value, both of which are preferably preset.

Described below with reference to FIGS. 6-12, is a further embodiment of the apparatus according to the invention.

It should be noted that although the numbering of the components of this embodiment is different, components which are functionally similar are, where possible, denoted by similar numerals starting with 1 instead of 2.

In this embodiment, the feed means 112 are associated with the tank 111 to withdraw the material SAP1 and are configured to move a preset flow quantity “Q” of material SAP1 along a feed direction “C”.

It should be noted that the feed means 112 are designed to move the material SAP1 along a feed front “F” having a preset width “L”, transversal to the feed direction “C”, and a preset thickness “S”.

In other words, the feed means 112 have a preset width (that is, a dimension at right angles to the feed direction “C”) which delimits (and is thus equal to) the aforementioned preset width “L” of the feed front “F”.

In the preferred embodiment, the feed means 112 comprise a conveyor 113 located at the base of the tank 111 to receive the absorbent polymer material SAP1 which is gravity-fed thereon.

Thus, the width of the conveyor 113 is equal to the preset width “L” of the feed front “F”.

Preferably, the conveyor 113 is defined by a substantially horizontal vibrating channel 113a.

In the preferred embodiment, the feed direction “C” of the feed means 112, that is, the axis of extension of the conveyor 113, is substantially parallel to the operating direction “A”.

The feed means 112 are operatively (and effectively) interposed between the tank 111 and the first feed means 101 by which the first web NW1 of non-woven fabric is fed.

It should be noted that the “working width” of the first web NW1 is greater than the width “L” of the feed front “F” determined by the feed means 112.

Thus, the working width of the first web NW1 is greater than the width of the feed means 112.

In this regard, according to the invention, the apparatus 110 comprises distribution means 114 which are interposed between the feed means 112 and the first feed means 101 and which are configured to adapt the width of the feed front “F” of the absorbent polymer material SAP1 to the “working width” of the first web NW1.

Thus, the distribution means 114 are interposed between the feed means 112, that is, the conveyor 113, and the drum or conveyor 103.

In the embodiment illustrated, the distribution means 114 are located at a height above the drum or conveyor 103.

In other words, the distribution means 114 are located at a greater distance from the machine base than the drum or conveyor 103. In light of this, the polymer material SAP1 is distributed at least partly by gravity (that is to say, it is allowed to drop).

The distribution means 114 are thus configured to receive the absorbent polymer material SAP1 from the feed means 112 and to distribute it on the first feed means 101, that is, on the drum or conveyor 103, uniformly and diffusely.

In this regard, the distribution means 114 comprise at least one diffuser element 115 extending along a respective axis “D” between an infeed portion 115a of it and an outfeed portion 115b of it.

The infeed portion 115a faces the feed means 112 to receive the preset flow quantity “Q” of absorbent polymer material SAP1.

The outfeed portion 115b faces the drum or conveyor 103 (at a height above the latter) and is provided with an opening 116 having a main dimension “D2out” transversal to the operating direction “A” of the drum or conveyor 103.

More precisely, the opening 116 of the outfeed portion 115b extends at right angles to the operating direction “A” of the drum or conveyor 103. In other words, the opening 116 of the outfeed portion 115b extends at right angles to the axis “D” of extension of the diffuser element 115.

It should be noted that the main dimension “D2out” of the opening 116 corresponds to the “working width” of the first web NW1.

The diffuser element 115 is configured to modify the feed front “F” of the absorbent polymer material SAP1 along its axis “D” by reducing the thickness and increasing the width (from the preset value to a value equal to the main dimension “D2out”) in order to distribute the absorbent polymer material SAP1 uniformly on the drum or conveyor 103 (that is, on the first web NW1) transversely to the operating direction “A”.

Advantageously, it is thus possible to distribute the absorbent polymer material SAP1 across the full width of the first web NW1 without having to modify the components upstream, that is to say, without modifying either the feed means 112 or the tank 111.

More in detail, the diffuser element 115 comprises a duct 117 extending between the infeed portion 115a and the outfeed portion 115b.

The duct extends between its inlet 117a, located at the infeed portion 115a, and its outlet 117b, corresponding to the opening 116.

The diffuser element 115 is shaped in such a way that the duct 117 has a transversal cross section “D”, defined by a first dimension “D1” and a second dimension “D2” which are at right angles to each other.

The first dimension “D1” decreases along the axis “D” to reduce the thickness “S” of the feed front “F” of the flow quantity “Q” of absorbent polymer material SAP1.

The second dimension “D2” increases to increase the width “L” of the feed front “F” of the flow quantity “Q” of absorbent polymer material SAP1.

It should be noted that the decrease in the first dimension “D1” is greater than the increase in the second dimension “D2”.

Thus, the cross section (transversal to the axis “D”) of the duct 117 decreases in size between the infeed portion 115a and the outfeed portion 115b of the diffuser element 115.

In other words, the cross section of the duct 117 (transversal to the axis “D”) decreases from the inlet 117a to the outlet 117b.

Moreover, to make distribution gradual and uniform, the duct 117 has a width (or second dimension “D2”) which increases from its inlet 117a to its outlet 117b.

Preferably, the duct 117 is shaped in such a way that the ratio between the first dimension “D1 in” of the infeed portion 115a and the first dimension “D1 out” of the outfeed portion 115b is greater than fifteen, and more preferably is around twenty.

Also, preferably, the ratio between the second dimension “D2out” of the outfeed portion 115b (that is, the width of the opening 116) and the second dimension “D2in” of the infeed portion 115a is between five and ten, and more preferably, is around seven.

More generally speaking, the ratio between the second dimension “D2out” of the outfeed portion 115b (that is, the width of the opening 116) and the width of the feed means 112 is between five and ten, and more preferably, is around seven.

In the preferred embodiment, the diffuser element 115 has a bottom wall 118 and a top wall 119 which converge towards each other from the infeed portion 115a to the outfeed portion 115b, thereby reducing the height of the duct 117.

In other words, the convergence of the bottom wall 118 and top wall 119 determines the aforementioned reduction in the first dimension “D1” of the duct 117.

Operatively, it is the top wall 119 which, as it converges towards the bottom wall 118, exerts pressure on the feed front “F” in such a way as to widen it.

It should be noted that in the embodiment illustrated, the bottom wall 118 is substantially horizontal.

In the embodiment illustrated, the bottom wall 118 and the top wall 119 are defined by a first plate 118a and a second plate 119a, respectively, trapezoidal in shape, which face each other and which are inclined at an angle to each other in such a way that the respective major bases, defining the outfeed portion 115b of the diffuser element 115, are proximal to the respective minor bases which define the infeed portion 115a.

Preferably, the top wall 119 makes with the bottom wall 118 an angle such that (that is, their degree of inclination relative to each other is such that) in the absence of movements of the diffuser element 115, the movement of the feed front “F” towards the outfeed portion 115b is stopped when the duct 117 is full to a preset extent.

In other words, the reduction of the cross section, and in particular of the first dimension “D1”, produces compression in the absorbent polymer material SAP1 which stops its forward motion.

In this regard, the distribution means 114 comprise a vibrating device 120 configured to shake the duct 117 (that is, the diffuser element 115) in order to re-start the forward motion of the feed front “F” so as to allow distribution along the second dimension “D2” of the duct 117.

In other words, the vibration imparted to the diffuser element 115 “loosens up” the absorbent polymer material SAP1 inside the duct 117, allowing material to be diffused along the second dimension “D2” and to be fed out through the outlet 117b (that is, through the opening 116).

In light of this, the apparatus 110 preferably comprises (or is associated with) a control unit 121 associated with the feed means 112 and with the vibrating device 120.

The control unit 121 is configured to activate the feed means 112 to supply the flow quantity “Q” of absorbent polymer material SAP1 to the diffuser element 115 until reaching the condition where it is full to the preset extent. The control unit 121 is programmed to activate the vibrating means 121 after, or concurrently with, the reaching of this condition, in order to re-start the forward motion of the feed front “F”.

Advantageously, this keeps the flow quantity “Q” constant and the distribution of the absorbent polymer material SAP1 uniform because the diffusing action applied by the diffusing element 115 is maximized. This is due mainly to the fact that after the absorbent polymer material SAP1 has been compacted in the duct 117, the vibrating action loosens up the absorbent polymer material SAP1 which, by reducing the first dimension “D1” of the duct 117, is widened out (or stretched out over a larger area) and distributed across the full outlet, that is, is made to fill the entire gap defined by the opening 116.

In other words, the control unit 121 is programmed to drive two successive steps when the machine is started, that is:
a first step of filling the duct where it drives only the feed means 112;
a second step of distributing the particulate polymer material SAP1, where it also drives the vibrating device 120.

Thus, both of the embodiments described above are applicable to a machine 100 according to the invention where, preferably, the dosing and depositing station 109 is equipped with two apparatuses 110 for distributing particulate material (that is, absorbent polymer material).

More precisely, in addition to the apparatus 110, 210 (in one of the two embodiments described up to now), the dosing and depositing station 109 comprises a further distribution apparatus 122, 310 (of the same kind).

Preferably, the further apparatus 122, 310 is configured to distribute on the first web NW1 a further particulate material SAP2.

Thus, in the embodiment illustrated, the further distribution apparatus 122, 310 is equipped with its own tank 123, 311, its own feed means 124, 312 and its own distribution means 125, 314 where the tank 123, 311 contains a further type of absorbent polymer material, which we shall call SAP2.

The use of two distinct absorbent polymer materials SAP1 and SAP2 allows differentiating the absorbent capacity in the different parts of the pad (that is, of the absorbent sanitary article).

Advantageously, the tank 123, 311, the feed means 124, 312 and the distribution means 125, 314 are structurally similar to those described above for the apparatus 110, 210.

Thus, the distribution means 125, 314 of the further apparatus 122, 310 are equipped with a diffuser element 126, 315 similar to the one described above and with a dedicated vibrating device 127, 320.

It should be noted that the two apparatuses 110, 122 or 210, 310 are preferably located in series along the operating direction “A” of the first feed means 101. More precisely, the respective diffuser means 115, 126 or 215, 315 are located one after the other in succession.

Preferably, the outfeed portions of the diffuser elements 122, 126 or 215, 315 at least partly face distinct zones of the first web NW1 in order to differentiate the absorption capacity of the absorbent sanitary article.

In effect, the first web NW1 has a middle portion “M” which extends along the operating direction “A” and which is interposed between two peripheral portions, or edge portions, “P”.

The diffuser element 115, 215 of the apparatus 110, 210 has an outfeed portion 115b, 215b having a width “L” which substantially corresponds to a “working width” of the first web NW1 in order to distribute the absorbent polymer material SAP1 both on the middle portion “M” and on the peripheral portions “P” of the first web NW1.

The diffuser element 126, 315 of the second apparatus 122, 310, on the other hand, has an outfeed portion 126b, 315b having a width “L” which substantially corresponds to a width of the middle portion “M” of the first web NW1.

Thus, the further absorbent polymer material SAP2 is distributed only in the central part of the absorbent sanitary article.

Advantageously, the absorption capacity of the pad can thus be differentiated in a simple and precise manner.

In light of this, the width of the outfeed portion 126b, 315b of the diffuser element 126, 315 of the further apparatus 122, 310 is smaller than the width of the outfeed portion 115b, 215b of the diffuser element 115, 215 of the apparatus 110, 210.

In alternative embodiments, there might be only one type of absorbent polymer material.

Thus, both of the apparatuses 110 and 122 or 210, 310 would be connected to a single tank.

In this event, the different absorption capacity of the zones of the pad is due mainly to the different quantities of the absorbent polymer materials SAP1, SAP2 in the different zones.

In a further alternative embodiment, there is a single distribution apparatus 110, 210.

To differentiate the distribution of absorbent polymer material SAP1 on the first web NW1, the diffuser element 115, 215 is equipped with movable longitudinal partitions (illustrated in only some embodiments but applicable to all embodiments) extending from the infeed portion 115a, 215a to the outfeed portion 115b, 215b in order to divide the duct into three channels, a middle channel and two lateral channels.

Preferably, the partitions are pivoted at the outfeed portion 115b, 215b to allow adjusting the flow quantity of polymer material entering each channel without varying the dimension (in particular, the second dimension) of the outfeed portion of each channel.

Advantageously, it would thus be possible to adjust the quantity of absorbent polymer material SAP1 without having to use two apparatuses but simply by controlling the movement of the movable partitions.

In order to hold down and feed the first web NW1 along the operating direction, the drum or conveyor 103 has a plurality cavities 128 formed along its periphery and suction means 129 associated with the cavities 128 and configured to produce a negative pressure at the cavities 128 themselves so that each cavity holds down a portion of the first web NW1.

The “held-down” portion defines a zone (or pocket) for receiving the absorbent polymer material SAP1, SAP2.

More precisely, the cavities 128 are formed on the peripheral surface 105a of the roller 105.

In the preferred embodiment, the peripheral surface 105a of the roller 105 has a plurality of angularly equispaced openings which can be coupled to suitable tiles 130 defining the cavities 128.

In the embodiment illustrated, the tiles 130 are defined by a net or mesh where the cavities are defined by the openings of the net or mesh.

Alternatively, the openings might be spaced further apart.

It should be noted that the suction means 129 are of a size such as to produce at each cavity 128, a negative pressure which can hold down the absorbent polymer material SAP1, SAP2 in the respective pocket.

More precisely, the negative pressure is such that all the absorbent polymer material SAP1, SAP2 interposed between two adjacent cavities 128 is transported into the respective pockets.

Thus, zones which are free of the polymer material SAP1, SAP2 are defined on the first web NW1.

In the embodiment illustrated, these zones correspond to the threads of the net and are therefore substantially rectilinear.

It should be noted that the coupling station 106, that is, the joining unit 107, is configured to join the second web NW2 to these free zones, thus optimizing the seal between the two.

The invention achieves the preset aims and brings important advantages.

In effect, thanks to the use of a distribution apparatus capable of uniformly widening the feed front of the material, it is possible to make absorbent sanitary articles in “cross-direction”, that is, by cutting the web in such a way that the shapes of the articles (in this case, of the pads) are at right angles to the operating direction, thereby increasing the productivity of the production plant as a whole.

Also, the use of a diffuser element capable of keeping the flow quantity constant and preventing accumulation of material increases the quality of the product and prevents the zones of absorption of body exudates from losing their effectiveness.

Moreover, the preferred embodiment, equipped with a duct which is convergent (in height) and divergent (in width), coupled to a vibrating device is particularly efficient at reduced costs, with considerable economic advantages both for the constructor of the machine and for the manufacturer of the absorbent sanitary articles.

1. An apparatus for distributing particulate material on a drum or conveyor movable along a respective operating direction, the apparatus comprising:
at least a tank containing particulate material and equipped with feed means for feeding the particulate material;
distribution means located downstream of the tank receive the particulate material from the feed means and configured to distribute the particulate material on the drum or conveyor;
wherein the distribution means comprise at least a diffuser element and a vibrating device configured to shake the diffuser element, where the diffuser element extends with its sliding plane along its axis between an infeed portion of it, facing the feed means to receive a preset flow quantity “Q” of particulate material, and an outfeed portion of it, of predetermined width, which can be positioned to face the drum or conveyor; the diffuser element being equipped, at an intermediate position between the infeed portion and the outfeed portion with at least one transversal partition at a predetermined distance from the sliding plane in order to form a neck which acts in combination with a vibration imparted by the vibrating device in such a way as to distribute the particulate material along the predetermined width.

2. The apparatus according to claim 1, wherein it comprises weight measuring means associated with the tank and configured to measure the weight thereof at predetermined intervals in order to make available a signal representing the consumption of the particulate material.

3. The apparatus according to claim 2, wherein the weight measuring means are also associated with distribution means in order to make available a signal representing the consumption of the particulate material in the entire apparatus.

4. The apparatus according to claim 2, wherein the weight measuring means comprise one or more load cells associated with the tank and/or with the distribution means.

5. The apparatus according to claim 2, wherein it comprises a control unit:
associated with the weight measuring means and with the vibrating device,
configured to receive from the weight measuring means the signal representing the consumption of the particulate material; and
configured to drive the vibrating device as a function of the signal representing the consumption of the particulate material.

6. The apparatus according to claim 5, wherein the control unit is programmed:
to compare the signal representing the consumption of the particulate material sent by the weight measuring means a preset reference value;
to generate an increment in the power of the vibrating device if the consumption of the particulate material is less than the preset reference value; and
to generate a decrease in the power of the vibrating device if the consumption of the particulate material is greater than the preset reference value.

7. The apparatus according to claim 1, wherein the feed means are at least partly defined by a bottom portion the tank having a release mouth of predetermined size and configured to release the particulate material by gravity into the infeed portion of the diffuser element.

8. The apparatus according to claim 7, wherein the width of the release mouth is smaller than the predetermined width the outfeed portion of the diffuser element.

9. The apparatus according to claim 1, wherein the transversal partition is movably connected to the diffuser element so that its position can be adjusted towards and/or away from the sliding plane as a function of a flow quantity of the particulate material and/or of the predetermined width of the outfeed portion of the diffuser element.

10. A machine for making absorbent sanitary articles comprising at least an impermeable outer layer and an absorbent pad superposed on the outer layer, where the machine comprises at least a feed line for feeding the outer web and a unit for forming the absorbent pad, the forming unit comprising:
first feed means for feeding a first web of non-woven fabric along the operating direction;
second feed means for feeding a second web of non-woven fabric;
a coupling station to the second web of non-woven fabric;
a dosing and depositing station, which is operatively located upstream of the coupling station and where at least one distributed quantity of absorbent polymer material is placed on the first web of non-woven fabric;
wherein the dosing and depositing station comprises an apparatus for distributing particulate material according to claim 1, where the first feed means are at least partly defined by a drum or conveyor.

11. The machine according to claim 10, wherein the dosing and depositing station comprises a further apparatus for distributing a further particulate material and equipped with its own diffuser element, where the diffuser elements of the distribution apparatus and of the further distribution apparatus have outfeed portions which at least partly face distinct zones of the first web in order to differentiate the absorption capacity of the absorbent sanitary article.

12. The machine according to claim 11, wherein the first web has a middle portion extends along the operating direction and which is interposed between two peripheral portions or edge portions; the diffuser element of the apparatus having an outfeed portion having a predetermined width substantially corresponding to a width of the first web in order to distribute the particulate material both on the middle portion and on the peripheral portions of the first web itself; the diffuser element of the further apparatus having an outfeed portion having a predetermined width substantially corresponding to a width of the middle portion of the first web and facing it in order to distribute the further particulate material on the middle portion.

13. The machine according to claim 10, wherein the drum or conveyor has a plurality of cavities along its periphery and suction means associated with the cavities and configured to produce a negative pressure at the cavities themselves so that each cavity holds down a portion of the first web to form a pocket for receiving the particulate material.

14. The machine according to claim 13, wherein the drum or conveyor is defined by a roller having a peripheral surface which is cylindrical and rotatable about a respective axis of rotation; the cavities being formed on the peripheral surface and being angularly equispaced.

15. The machine according to claim 13, wherein the suction means are of a size such as to produce at each cavity, a negative pressure such that the particulate material interposed between two adjacent cavities is transported into the respective pockets, defining on the first web zones which are free of the particulate material and which can be joined to the second web.
